SEQUENCE LISTING (9 sequences)

SEQ ID NO: 1 - protein, 12 aa, Homo sapiens
MADNFSLHDALS

SEQ ID NO: 2 - protein, 14 aa, Homo sapiens
MADNFSLHDALSGS

SEQ ID NO: 3 - protein, 11 aa, Homo sapiens
GNPNPQGWPGA

SEQ ID NO: 4 - protein, 10 aa, Homo sapiens
WGNQPAGAGG

SEQ ID NO: 5 - protein, 18 aa, Homo sapiens
YPGQAPPGAYPGQAPPGA

SEQ ID NO: 6 - protein, 16 aa, Homo sapiens
YPGAPGAYPGAPAPGV

SEQ ID NO: 7 - protein, 19 aa, Homo sapiens
YPGAPAPGVYPGPPSGPGA

SEQ ID NO: 8 - protein, 12 aa, Homo sapiens
YPSSGQPSATGA

SEQ ID NO: 9 - protein, 31 aa, Homo sapiens
YPGAPAPGVYPGPPSGPGAYPSSGQPSATGA

(19) United States
(12) Patent Application Publication
(10) Pub. No.: US 2013/0344517 A1
(43) Pub. Date: Dec. 26, 2013

(54) USE OF GALECTIN-3 FOR RISK ASSESSMENT AND DETECTION OF PREECLAMPSIA AND RELATED CONDITIONS

(71) Applicant: BG Medicine, Inc., Waltham, MA (US)

(72) Inventor: Pieter Muntendam, Boxford, MA (US)

(73) Assignee: BG Medicine, Inc., Waltham, MA (US)

(21) Appl. No.: 13/735,895

(22) Filed: Jan. 7, 2013

Related U.S. Application Data
(60) Provisional application No. 61/583,885, filed on Jan. 6, 2012.

(51) Int. Cl. G01N 33/68

(52) U.S. Cl. CPC G01N 33/689
USPC 435/7.94; 435/7.92; 436/501

(57) ABSTRACT

Described herein are materials and methods for predicting and/or monitoring preeclampsia and related conditions, including eclampsia, HELLP (hemolysis, elevated liver enzymes, and low platelet count) syndrome, left ventricular dysfunction, and heart failure, in a pregnant or post-partum woman. More specifically, use of the endogenous protein galectin-3 in predicting and/or monitoring preeclampsia and related conditions in a pregnant or post-partum woman is described. Also described are methods of preventing and/or treating preeclampsia and related conditions by inhibiting galectin-3.

### REFERENCE TO RELATED APPLICATIONS

This application claims the benefit of and priority to U.S. Provisional Patent Application No. 61/583,885, filed Jan. 6, 2012, the complete disclosure of which is incorporated by reference herein.

### BACKGROUND

Preeclampsia and its related conditions, such as eclampsia, HELLP syndrome (hemolysis, elevated liver enzymes, and low platelet count syndrome), left ventricle dysfunction, and heart failure, are life-threatening complications of pregnancy. In the United States, these conditions afflict approximately 5-8% of all pregnant women. Preeclampsia is characterized by hypertension and proteinuria. Most women that develop preeclampsia subsequently do not develop one of the related conditions described above. However, in some cases, a preeclampsic woman may go on to develop eclampsia, which is characterized by seizures or convulsions. Although preeclampsia generally appears first, one or more of the related conditions may develop concurrently with preeclampsia. For example, HELLP symptoms are typically observed after diagnosis of preeclampsia, but HELLP symptoms may sometimes be the first indication of preeclampsia. Preeclampsia and related conditions can develop gradually or suddenly, may develop at any time during pregnancy, and can persist for up to about six weeks post-partum.

Symptoms of the conditions described above may be treated with medications, for example antihypertensives, corticosteroids, or anticonvulsive medications; however, such treatments merely alleviate the symptoms and do not treat the underlying cause of the symptoms. The only presently known cure for preeclampsia and its related conditions is delivery of the baby. Depending on the severity of the woman's condition, preterm induction of labor or caesarean section may be necessary. Preterm delivery, however, presents a risk of death to the baby depending on the birth weight of the baby and the stage of development of the baby's lungs and other organs.

Current methods for predicting which women will acquire preeclampsia and which women will not rely on risk factors such as age, obesity, and past history of diabetes, hypertension, or kidney disease and assays of biomarkers such as angiogenic factors (Scazzocchio (2011) Curr. Opin. Obstet. Gynecol. 23:65-71). However, current tests that attempt to predict preeclampsia on the basis of measuring levels of biomarkers are limited by poor predictive accuracy and/or the need to combine a plurality of tests using statistical models.

### SUMMARY OF THE INVENTION

Levels of the human protein galectin-3 can be used to predict and/or monitor preeclampsia and related conditions. These conditions may include, for example, eclampsia, HELLP (hemolysis, elevated liver enzymes, and low platelet count) syndrome, left ventricular dysfunction, and/or heart failure, in a pregnant or post-partum woman. A pregnant or post-partum woman's galectin-3 concentration or activity in a bodily fluid (e.g., blood, plasma, or serum) can be determined to evaluate risk for development (such as initial development, or progression) of preeclampsia and/or related conditions. In addition, a pregnant or post-partum woman's galectin-3 blood concentration or activity can be monitored over the course of a treatment for preeclampsia and/or related conditions to determine the efficacy of treatment. Furthermore, inhibition of galectin-3 may be used to treat preeclampsia and/or related conditions.

In one embodiment, methods for assessing preeclampsia or eclampsia risk in a pregnant or post-partum woman are provided. For example, a galectin-3 level in a sample comprising blood, serum, or plasma from the pregnant or post-partum woman can be measured thereby to determine the presence or absence of a galectin-3 level indicative of a preeclampsia or eclampsia risk.

In another embodiment, methods of assessing hemolysis, elevated liver enzymes, low platelets (HELLP) risk in a pregnant or post-partum woman are provided. For example, a galectin-3 level in a sample comprising blood, serum, or plasma from the pregnant or post-partum woman can be measured thereby to determine the presence or absence of a galectin-3 level indicative of a HELLP risk.

In yet another embodiment, methods of assessing left ventricle dysfunction risk in a pregnant or post-partum woman are provided. For example, a galectin-3 level in a sample comprising blood, serum, or plasma from the pregnant or post-partum woman can be measured thereby to determine the presence or absence of a galectin-3 level indicative of a left ventricle dysfunction risk.

In still another embodiment, methods of assessing heart failure risk in a pregnant or post-partum woman are provided. For example, a galectin-3 level in a sample comprising blood, serum, or plasma from the pregnant or post-partum woman thereby to determine the presence or absence of a galectin-3 level indicative of a heart failure risk.

In yet another embodiment, methods of assessing a pregnant or post-partum woman for or treated with anti-galectin-3 therapy are provided. For example, a galectin-3 level in a sample from the pregnant or post-partum woman may be measured, thereby to determine the presence or absence of a galectin-3 level indicative of responsiveness to, or prognosis following, anti-galectin-3 therapy. The anti-galectin-3 therapy may include administering a compound in an amount sufficient to at least partially alleviate a symptom of eclampsia; preeclampsia; hemolysis, elevated liver enzymes, low platelets (HELLP); left ventricle dysfunction; and/or heart failure, where the compound binds to galectin-3. In certain embodiments, the anti-galectin-3 therapy may include administering a carbohydrate capable of binding to galectin-3 to the pregnant or post-partum woman. For example, the carbohydrate may be a pectin or pectin fragment.

In still another embodiment, methods of identifying a pregnant or post-partum woman as a candidate for personalized nutritional advice are provided. For example, a galectin-3 level in a sample from the pregnant or post-partum woman may be measured, thereby to determine the presence or absence of a galectin-3 level indicative of an increased potential to benefit from personalized nutritional advice.

In yet another embodiment, methods of monitoring development or progression of preeclampsia or eclampsia in a pregnant or post-partum woman are provided. For example, a galectin-3 level in a sample from the pregnant or post-partum woman may be measured, thereby to determine the presence or absence of a galectin-3 level indicative of the development or progression of preeclampsia or eclampsia.

In still another embodiment, methods of monitoring development or progression of hemolysis, elevated liver enzymes, low platelets (HELLP) in a pregnant or post-partum woman are provided. For example, a galectin-3 level in a sample from the pregnant or post-partum woman may be measured, thereby to determine the presence or absence of a galectin-3 level indicative of the development or progression of HELLP.

In yet another embodiment, methods of monitoring development or progression of left ventricle dysfunction in a pregnant or post-partum woman are provided. For example, a galectin-3 level in a sample from the pregnant or post-partum woman may be measured, thereby to determine the presence or absence of a galectin-3 level indicative of the development or progression of left ventricle dysfunction.

In still another embodiment, methods of monitoring development or progression of heart failure in a pregnant or post-partum woman are provided. For example, a galectin-3 level in a sample from the pregnant or post-partum woman may be measured, thereby to determine the presence or absence of a galectin-3 level indicative of the development or progression of heart disease.

In yet another embodiment, methods of treating eclampsia; preeclampsia; hemolysis, elevated liver enzymes, low platelets (HELLP); left ventricle dysfunction; and/or heart failure in a pregnant woman are provided. For example, the method may include delivering the pregnant woman's baby if a galectin-3 level or change in galectin-3 level in a blood, serum, or plasma sample from the pregnant woman exceeds a minimum threshold. Delivering the pregnant woman's baby may include, for example, inducing labor, performing a caesarean section, and/or administering a compound selected from the group consisting of oxytocin, a prostaglandin, and misoprostal. In some instances, the prostaglandin may be dinoprostone.

In still another embodiment, methods of treating preeclampsia in a pregnant or post-partum woman are provided. For example, galectin-3 in a preeclamptic pregnant or post-partum woman identified as having elevated circulatory levels of galectin-3 may be inhibited to at least partially alleviate a symptom of preeclampsia. The symptom may be, for example, hypertension. In some embodiments, the pregnant or post-partum woman's resting systolic and/or diastolic blood pressure may be reduced by at least about 5 mm Hg. The pregnant or post-partum woman's resting blood pressure prior to treatment may be at least about 140/90 mm Hg or at least about 160/110 mm Hg. In another embodiment, the symptom may be proteinuria. The proteinuria prior to treatment may be at least about 300 mg of protein in a 24 hour collection period or at least about 5 g of protein in a 24 hour collection period. In some instances, the proteinuria may be reduced by at least about 50 mg in a 24 hour collection period.

In yet another embodiment, methods of treating eclampsia in a pregnant or post-partum woman are provided. For example, galectin-3 in an eclamptic pregnant or post-partum woman identified as having elevated circulatory levels of galectin-3 may be inhibited to at least partially alleviate convulsions.

In still another embodiment, methods of treating hemolysis, elevated liver enzymes, low platelets (HELLP) in a pregnant or post-partum woman are provided. For example, galectin-3 in a pregnant or post-partum woman identified as having elevated circulatory levels of galectin-3 may be inhibited to at least partially alleviate one or more of hemolysis, elevated liver enzymes, and low platelet count. The low platelet count prior to treatment may correspond, for example, to a platelet count of less than about 100,000/mm³, between about 50,000/mm³ and about 100,000/mm³, or less than about 50,000/mm³. In some embodiments, the elevated liver enzymes may be serum aspartate aminotransferase and lactate dehydrogenase. The serum aspartate aminotransferase may have, for example, a level prior to treatment of greater than about 70 U/L or greater than about 600 U/L. In certain embodiments, the hemolysis may be characterized by an abnormal peripheral smear. In some cases, the hemolysis may be characterized by a lactate dehydrogenase level prior to treatment of greater than about 600 U/L. The hemolysis may, in some embodiments, be characterized by a bilirubin level prior to treatment of greater than about 1.2 mg/dL.

In a pregnant or post-partum woman, the galectin-3 level can be measured during the first trimester of pregnancy, the second trimester of pregnancy, or during the third trimester of pregnancy. In some cases, the galectin-3 level may be measured post-term or post-partum. In some embodiments, a subsequent galectin-3 level can be measured in a subsequent sample from the pregnant or post-partum woman, thereby to detect a change in galectin-3 levels. A galectin-3 level can also be measured in a sample from the woman prior to conception, thereby to detect a change in galectin-3 levels.

The galectin-3 levels can change over time. For instance, the change in galectin-3 levels may be an increase in galectin-3 levels over time. In some embodiments, the pregnant or post-partum woman may have a galectin-3 level determined to be above a minimum threshold. In other embodiments, the change in galectin-3 level may be above a minimum threshold. For example, the minimum threshold may be more than 6 ng/ml, more than 10 ng/ml, between 6 and 19 ng/ml, between 10 and 15 ng/ml, between 15 and 20 ng/ml, between 20 and 25 ng/ml, between 25 and 30 ng/ml, or more than 30 ng/ml. The measured galectin-3 level may be determined, for example, by an immunoassay.

### BRIEF DESCRIPTION OF THE DRAWINGS

FIG. 1 shows a plot depicting distribution of galectin-3 levels for a subject group having preeclampsia and a subject group not having preeclampsia.

FIG. 2 shows a plot depicting a receiver-operating characteristic curve for discrimination of preeclampsia subjects and no preeclampsia subjects. The dashed diagonal line represents a slope of unity.

Other aspects, embodiments, and features will be apparent from the following detailed description when considered in conjunction with the accompanying figures.

### DETAILED DESCRIPTION OF THE INVENTION

Applicants have invented a method of predicting and/or monitoring preeclampsia and related conditions in a pregnant or post-partum woman. Applicants have discovered that measuring the circulating galectin-3 levels in a pregnant or post-partum woman can be used to identify those woman who are at risk of developing preeclampsia and/or related conditions, such as eclampsia, HELLP (i.e., hemolysis, elevated liver enzymes, and low platelet count), left ventricle dysfunction, and heart failure. At-risk pregnant or post-partum woman may be monitored proactively for development of preeclampsia so as, for example, to allow intervention before serious complications result. Additionally, preventative treatment of the pregnant or post-partum woman may be initiated to delay or inhibit development of preeclampsia and/or related conditions. Applicants have further discovered that measuring galectin-3 in pregnant or post-partum women having or at risk of developing preeclampsia and/or related conditions provides an ongoing indication of development or progression of the condition and/or of the continued propriety of the course of treatment.

### DEFINITIONS

As used herein, “preeclampsia” refers to a disorder characterized by hypertension with proteinuria or edema, or both, glomerular dysfunction, brain edema, liver edema, or coagulation abnormalities due to pregnancy or the influence of a recent pregnancy. Preeclampsia generally occurs after the 20th week of gestation. Preeclampsia is generally defined as some combination of the following symptoms: (1) a blood pressure of at least 140/90 mm Hg after 20 weeks gestation (generally measured on two occasions, 4-168 hours apart), (2) new onset proteinuria (at least 1+ by dipstik on urinanalysis, at least 0.3 g of protein in a 24-hour urine collection period, or a single random urine sample having a protein/creatinine ratio greater than 0.3), and (3) resolution of hypertension and proteinuria by 12 weeks postpartum. Severe preeclampsia is generally defined as (1) a blood pressure of at least 160/110 mm Hg (generally measured on two occasions, 4-6 hours apart) or (2) proteinuria characterized by a measurement of 5 g or more protein in a 24-hour urine collection or two random urine specimens with at least 3+ protein by dipstick. Other elements of severe preeclampsia may include in-utero growth restriction (IUGR) in less than the 10% percentile according to the U.S. demographics, persistent neurologic symptoms (headache, visual disturbances), epigastric pain, oliguria (less than 500 mL/24 h), serum creatinine greater than 1.0 mg/dL, elevated liver enzymes (greater than two times normal), and thrombocytopenia (<100,000 cells/μL). In preeclampsia, hypertension and proteinuria generally occur within seven days of each other.

Occasionally, preeclampsia can lead to the development of convulsions or seizures. This severe form of the syndrome is referred to as “eclampsia.” Preeclampsia and eclampsia can also include dysfunction or damage to several organs or tissues such as the liver (e.g., hepatocellular damage, periportal necrosis), the central nervous system (e.g., cerebral edema and cerebral hemorrhage), the heart (e.g., left ventricular dysfunction (LVD) or heart failure), and the kidneys (e.g., renal dysfunction, glomerular endotheliosis, or hypertrophy).

Hemolysis, elevated liver enzymes, low platelets syndrome (referred to as “HELLP” syndrome) may also develop due to pregnancy or the influence of a recent pregnancy. HELLP is characterized by evidence of thrombocytopenia (i.e., less than 100,000 platelets/mm³), increased lactate dehydrogenase (LDH) (i.e., greater than 600 U/L) and increased aspartate aminotransferase (AST) (i.e., greater than 70 U/L). Thrombocytopenia may be mild (less than 100,000 platelets/mm³), moderate (between 50,000 platelets/mm³ and 100,000 platelets/mm³), or severe (less than 50,000 platelets/mm³). Hemolysis is characterized by a bilirubin level of greater than 1.2 mg/dL, LDH greater than 600 U/L, AST greater than 70 U/L and/or an abnormal peripheral smear. Hypertension, proteinuria, HELLP syndrome, and eclampsia can occur simultaneously or only one symptom at a time.

The terms “heart failure,” “HF,” “congestive heart failure,” or “CHF” as used herein, refer to the complex clinical syndrome that impairs the ability of the ventricle to fill with or eject blood. Any structural or functional cardiac disorder can cause HF, with the majority of HF patients having impaired left ventricular (LV) myocardial function (i.e., left ventricular dysfunction (LVD)). Cardiomyopathy may also lead to HF. Symptoms of HF include dyspnea (shortness of breath), fatigue, and fluid retention. The American Heart Association (AHA) has identified 4 stages in the progression or development of HF. Patients in stages A and B show clear risk factors but have not yet developed HF. Patients in stages C and D currently exhibit or in the past have exhibited symptoms of HF. For example, Stage A patients are those with risk factors such as coronary artery disease, hypertension or diabetes mellitus who do not show impaired left ventricular (LV) function. Stage B patients are asymptomatic, but have cardiac structural abnormalities or remodeling, such as impaired LV function, hypertrophy or geometric chamber distortion. Stage C patients have cardiac abnormalities and are symptomatic. Stage D patients have refractory HF in which they exhibit symptoms despite maximal medical treatment. They are typically recurrently hospitalized or unable to leave the hospital without specialized intervention.

Galectin-3 (GenBank Accession Nos.: NC_{—}000014.7 (gene) and NP_{—}002297.2 (protein)) is one of 15 mammalian beta galactoside-binding lectins, or “galectins,” characterized by their galactose-specific binding. Galectin-3 has variously been referred to in the literature as LGALS3, MAC-2 antigen, Carbohydrate binding protein (CBP)-35, laminin binding protein, galactose-specific lectin 3, mL-34, L-29, hL-31, epsilon BP, and IgE-binding protein. Galectin-3 is composed of a carboxyl-terminal carbohydrate recognition domain (CRD) and amino-terminal tandem repeats (Liu, F.-T. (2000) Role of galectin-3 in inflammation. In Lectins and Pathology. M. Caron and D. Seve, eds. Harwood Academic Publishers, Amsterdam, The Netherlands, p. 51; Liu, F.-T. et al. (1995) Am. J. Pathol. 147:1016). Galectin-3 normally distributes in epithelia of many organs and various inflammatory cells, including macrophages as well as dendritic cells and Kupffer cells (Flotte, T. J. et al. (1983) Am. J. Pathol. 111:112). Galectin-3 has also been detected in the placenta, for example, in trophoblastic tissue (van den Brule, F. A. et al. (1994) Biochem. Biophys. Res. Commun. 201:388).

### Detection of Galectin-3

Described herein are methods for monitoring and/or predicting risk of developing preeclampsia and related conditions (e.g., eclampsia, HELLP, left ventricular dysfunction, and HF) in a pregnant or post-partum woman by measuring the level of galectin-3. Many methods for detecting a protein of interest, with or without quantitation, are well known and can be used. Examples of such assays are described below and can include, for example, immunoassays, chromatographic methods, and mass spectroscopy. Such assays can be performed on any biological sample including, among others, blood, plasma, and serum. Accordingly, multiple assays can be used to detect galectin-3, and samples can be analyzed from one or more sources.

In some embodiments, the concentration of galectin-3 may be quantitated in a bodily fluid sample using a pair of binding moieties that bind specifically to N-terminal portions of galectin-3. A “binding moiety” refers to a molecule that binds or interacts selectively or preferentially with a polypeptide or peptide. Examples of binding moieties include, but are not limited to, proteins, such as antibodies, galectin binding protein (GBP) interaction fusion protein, peptide aptamers, avimers, Fabs, sFvs, Adnectins and Affibody® ligands; nucleic acids, such as DNA and RNA (including nucleotide aptamers), and lipids, such as membrane lipids.

In certain embodiments, detection of the concentration of galectin-3 in a clinical sample, such as serum, for the diagnosis of preeclampsia and/or related conditions may be made. The test sample used in the detection of galectin-3 can be any body fluid or tissue sample, including, but not limited to, whole blood, serum, plasma, urine, amniotic fluid, or placenta biopsy, and less preferably gastric juices, bile, saliva, sweat, spinal fluids, stool, lymph, or muscle biopsy. In a preferred embodiment, the sample is a blood sample. In another embodiment, the sample is a plasma sample. Serum samples may also be used. Furthermore, the body fluids may be either processed (e.g., serum) or unprocessed. Methods of obtaining a body fluid from a subject are known to those skilled in the art.

In some embodiments, galectin-3 may be detected and quantified using a “sandwich” assay. In this embodiment, two molecules (“binding moieties”) such as monoclonal antibodies that specifically bind to non-overlapping sites (“epitopes”) on the N-terminus of galectin-3 are used. Typically, one binding moiety is immobilized on a solid surface where it binds with and captures galectin-3. This first binding moiety is therefore also referred to herein as the capture binding moiety. A second binding moiety is detectably labeled, for example, with a fluorophore, enzyme, or colored particle, such that binding of the second binding moiety to the galectin-3-complex indicates that galectin-3 has been captured. The intensity of the signal is proportional to the concentration of galectin-3 in the sample. The second binding moiety is therefore also referred to herein as the detection binding moiety or label binding moiety. A binding moiety can be any type of molecule, as long as it specifically binds to a portion of the N-terminus of galectin-3. In a preferred embodiment, the binding moieties used are monoclonal anti-galectin-3 antibodies, i.e., monoclonals raised against or otherwise selected to bind to separate portions of the N-terminal 113 amino acids of galectin-3.

Such assay procedures can be referred to as two-site immunometric assay methods, “sandwich” methods or (when antibodies are the binders) “sandwich immunoassays.” As is known in the art, the capture and detection antibodies can be contacted with the test sample simultaneously or sequentially. Sequential methods, sometimes referred to as the “forward” method, can be accomplished by incubating the capture antibody with the sample, and adding the labeled detection antibody at a predetermined time thereafter. Alternatively, the labeled detection antibody can be incubated with the sample first and then the sample can be exposed to the capture antibody (sometimes referred to as the “reverse” method). After any necessary incubation(s), which may be of short duration, the label is detected and may also be measured. Such assays may be implemented in many specific formats known to those of skill in the art, including through use of various high throughput clinical laboratory analyzers or with point of care or home testing devices.

In one embodiment, a lateral flow device may be used in the sandwich format, wherein the presence of galectin-3 above a baseline sensitivity level in a biological sample will permit formation of a sandwich interaction upstream of or at the capture zone in the lateral flow assay. See, for example, U.S. Pat. No. 6,485,982. The capture zone as used herein may contain capture binding moieties such as antibody molecules, suitable for capturing galectin-3, or immobilized avidin or the like for capture of a biotinylated complex. See, for example, U.S. Pat. No. 6,319,676. The device may also incorporate a luminescent label suitable for capture in the capture zone, the concentration of galectin 3 being proportional to the intensity of the signal at the capture site. Suitable labels include fluorescent labels immobilized on polystyrene microspheres. Colored particles also may be used.

Other assay formats that may be used in the methods of the invention include, but are not limited to, flow-through devices. See, for example, U.S. Pat. No. 4,632,901. In a flow-through assay, one binding moiety (for example, an antibody) is immobilized to a defined area on a membrane surface. This membrane is then overlaid on an absorbent layer that acts as a reservoir to pump sample volume through the device. Following immobilization, the remaining protein-binding sites on the membrane are blocked to minimize non-specific interactions. In operation, a biological sample is added to the membrane and filters through, allowing any analyte specific to the antibody in the sample to bind to the immobilized antibody. In a second step, a labeled secondary antibody may be added or released that reacts with captured marker to complete the sandwich. Alternatively, the secondary antibody can be mixed with the sample and added in a single step. If galectin-3 is present, a colored spot develops on the surface of the membrane.

The most common enzyme immunoassay is the “Enzyme-Linked Immunosorbent Assay (ELISA).” ELISA is a technique for detecting and measuring the concentration of an antigen using a labeled (e.g., enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984), J. Clin. Chem. Clin. Biochem. 22:895-904.

In a “sandwich ELISA,” an antibody (e.g., anti-galectin-3) is linked to a solid phase (i.e., a microtiter plate) and exposed to a biological sample containing antigen (e.g., galectin-3). The solid phase is then washed to remove unbound antigen. A labeled antibody (e.g., enzyme linked) is then bound to the bound antigen, forming an antibody-antigen-antibody sandwich. Examples of enzymes that can be linked to the antibody are alkaline phosphatase, horseradish peroxidase, luciferase, urease, and β-galactosidase. The enzyme-linked antibody reacts with a substrate to generate a colored reaction product that can be measured. This measurement can be used to derive the concentration of galectin-3 present in a sample, for example, by comparing the measurement to a galectin-3 standard curve. Galectin-3 concentration (e.g., blood concentration) in a sample from a subject (e.g., a pregnant or post-partum woman) may be determined to be above or below a threshold or within a target range. The threshold may be in the range of, for example, about 0-15 ng/mL; about 0-20 ng/mL; about 6-19 ng/mL; about 6-12 ng/mL; about 7-12 ng/mL; about 8-12 ng/mL; about 9-12 ng/mL; about 10-12 ng/mL; about 5-10 ng/mL; about 10-15 ng/mL; about 15-20 ng/mL; about 20-25 ng/mL; about 25-30 ng/mL; about 30-35 ng/mL, or about 35-40 ng/mL. In some instances, the minimum threshold may be more than 6 ng/mL, more than 7 ng/mL, more than 8 ng/mL, more than 9 ng/mL, more than 10 ng/mL, more than 11 ng/mL, more than 12 ng/mL, more than 13 ng/mL, more than 14 ng/mL, more than 15 ng/mL, more than 16 ng/mL, more than 17 ng/mL, more than 18 ng/mL, more than 19 ng/mL, more than 20 ng/mL, or more than 30 ng/mL. In some cases, the galectin-3 blood concentration may be determined to be below a maximum threshold. For example, the maximum threshold may be below about 70 ng/mL, below about 60 ng/mL, or below about 40 ng/mL. The maximum threshold may be between about 30 and about 40 ng/mL, between about 25 and about 30 ng/mL, between about 20 and about 25 ng/mL, or between about 15 and about 20 ng/mL.

Any of the immunoassays described herein suitable for use with the kits and methods can also use any binding moiety in the place of an antibody.

### Binding Moieties

In a preferred embodiment of the invention, anti-galectin-3 antibodies, preferably monoclonal antibodies, are used as binding moieties. However, it should also be understood that the binding moieties described below may also be administered as galectin-3 inhibitors.

In preferred embodiments of the invention, monoclonal antibodies are used. A monoclonal antibody refers to an antibody that is derived from a single clone, including any eukaryotic, prokaryotic, or phage clone. The monoclonal antibody may comprise, or consist of, two proteins, i.e., heavy and light chains. The monoclonal antibody can be prepared using one of a wide variety of techniques known in the art including the use of hybridoma, recombinant, and phage display technologies, or a combination thereof.

Anti-galectin-3 monoclonal antibodies may be prepared using any known methodology, including the seminal hybridoma methods, such as those described by Kohler and Milstein (1975), Nature. 256:495. In a hybridoma method, a mouse, hamster, or other appropriate host animal is immunized with an immunizing agent to elicit lymphocytes that produce or are capable of producing antibodies that will specifically bind to the immunizing agent. Alternatively, the lymphocytes may be immunized in vitro.

The immunizing agent will typically include at least a portion of the galectin-3 polypeptide or a fusion protein thereof. For example, synthetic polypeptide or recombinant polypeptide comprising any galectin-3 N-terminal epitopes may be used as an immunizing agent. Exemplary N-terminal epitopes include, but are not limited to, MADNFSLHDALS (SEQ ID NO:1), MADNFSLHDALSGS (SEQ ID NO:2), GNPNPQGWPGA (SEQ ID NO:3), WGNQPAGAGG (SEQ ID NO:4), YPGQAPPGAYPGQAPPGA (SEQ ID NO:5), YPGAPGAYPGAPAPGV (SEQ ID NO:6), YPGAPAPGVYPGPPSGPGA (SEQ ID NO:7), YPSSGQPSATGA (SEQ ID NO:8). A fusion protein may be made by fusing a polypeptide to a carrier protein, for example, keyhole limpet hemocyanin (KLH, EMD Biosciences, San Diego, Calif.), BSA (EMD Biosciences, San Diego, Calif.), or ovalbumin (Pierce, Rockford, Ill.). The immunizing agent may be administered to a mammal with or without adjuvant according to any of a variety of standard methods. The immunizing agent may be administered only once, but is preferably administered more than once according to standard boosting schedules.

Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell population which is screened for species having appropriate specificity and affinity to epitopes on the N-terminal portion of galectin-3 (Goding, (1986) Monoclonal Antibodies: Principles and Practice, Academic Press, pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. (1984) Immunol., 133:3001; Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the N-terminus of galectin-3, e.g., by screening with a labeled galectin-3 N-terminal polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard (1980), Anal. Biochem., 107:220. Various analysis protocols to determine binding specificity are available commercially as kits or as a service.

Monoclonal antibodies also may be made by recombinant DNA methods, such as those described in U.S. Pat. No. 4,816,567. DNA encoding suitable monoclonal antibodies can be isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al., (1984) Proc. Natl. Acad. Sci. USA, 81:6851) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

Antibodies can also be produced using phage display libraries (Hoogenboom and Winter (1991), J. Mol. Biol. 227:381; Marks et al. (1991), J. Mol. Biol., 222:581). The techniques of Cole et al. and Boerner et al. are also available for the preparation of monoclonal antibodies (Cole et al. (1985), Monoclonal Antibodies and Cancer Therapy, Alan R. Liss, p. 77 and Boerner et al. (1991), J. Immunol., 147(1):86-95). Similarly, antibodies can be made by introducing of immunoglobulin loci into transgenic animals, e.g., mice in which the endogenous immunoglobulin genes have been partially or completely inactivated.

The antibodies may also be affinity matured using known selection and/or mutagenesis methods as described above. Preferred affinity matured antibodies have an affinity which is five times, more preferably 10 times, even more preferably 20 or 30 times greater than the starting antibody from which the matured antibody is prepared. In a particularly preferred embodiment, the antibodies used to detect galectin-3 are monoclonal antibodies, for example, M3/38, 9H3.2, and 87B5. M3/38 detects a linear epitope (YPGQAPPGAYPGQAPPGA (SEQ ID NO:5)) on the N-terminus of galectin-3. M3/38 was prepared from the supernatant of the rat hybridoma M3/38.1.2.8 HL.2, a clone of which can be found in the American Type Culture Collection with ATCC® number TIB-166. 9H3.2 detects a linear epitope (MADNFSLHDALSGS (SEQ ID NO:2) at the extreme N-terminus of galectin-3. 9H3.2 is a mouse monoclonal IgG, affinity purified using protein A. 9H3.2 is available from Millipore (Millipore, 290 Concord Road, Billerica, Mass. 01821, USA), catalog no.: MAB4033. 87B5 detects a non-linear epitope comprising portions of GNPNPQGWPGA (SEQ ID NO:3) and YPGAPAPGVYPGPPSGPGAYPSSGQPSATGA (SEQ ID NO:9). 87B5 was prepared from the mouse-mouse hybridoma (X63-Ag8.653×BALB/c mouse spleen cells) clone 87B5, and is an IgG2a that was affinity purified using Protein A. 87B5 is available from Immuno-Biological Laboratories (IBL, 8201 Central Ave NE, Suite P, Minneapolis, Minn. 55432 USA).

In a currently preferred embodiment, the capture binding moiety is the anti-galectin-3 monoclonal antibody, M3/38 and the labeled detection binding moiety is a second anti-galectin-3 monoclonal antibody, 87B5. The given designations for these antibodies are not limiting. In another embodiment, the capture antibody is 9H3.2 and the labeled detection binding moiety is M3/38. Other antibodies which recognize the epitopes described above also may be used.

Other binding moieties may be used with the methods and kits of the present invention. Examples of binding moieties include, but are not limited to, proteins, peptide aptamers, avimers, Adnectins and Affibody° ligands; nucleic acids, such as DNA and RNA (including nucleotide aptamers), and lipids, such as membrane lipids.

### Alternate Forms of Galectin-3

Galectin-3 may exist in a sample in a plurality of different forms characterized by detectably different masses. These forms can result from pre-translational modifications, post-translational modifications or both. Pre-translational modified forms include allelic variants, splice variants, and RNA-editing forms. Post-translationally modified forms include forms resulting from, among other things, proteolytic cleavage (e.g., fragments of a parent protein), complexation, glycosylation, phosphorylation, lipidation, oxidation, methylation, cystinylation, sulphonation and acetylation. Modified forms of galectin-3, as long as they retain the relevant N-terminal epitopes, may be detected according to the methods of the present invention.

### Diagnostic and Prognostic Uses

A galectin-3 assay can be used to identify pregnant or post-partum women at risk for developing or having preeclampsia and/or a related condition (e.g., hypertension, proteinuria, eclampsia, HELLP, LVD, HF, or the like). In another embodiment, galectin-3 may be used as a diagnostic marker to determine the presence, stage, or severity of preeclampsia and/or a related condition in a pregnant or post-partum woman or to predict her prognosis by measuring the concentration of galectin-3 in a sample and comparing this result to data correlating galectin-3 concentration with severity or stage of preeclampsia and/or a related condition. Methods of diagnosis and/or predicting prognosis described herein may be combined with other methods for diagnosis and/or predicting prognosis commonly used in the art, such as physical examination (e.g., to assess for edema and/or weight gain), urinalysis (e.g., to screen for proteinuria), blood tests, complete blood count, platelet count, blood clotting factors, bilirubin, creatinine, hematocrit, uric acid, serum electrolytes, glycohemoglobin and blood lipids, tests of renal and hepatic function (e.g., ADH and/or LST levels), tests of thyroid function, a chest radiograph, a 12-lead electrocardiogram, blood tests for biomarkers such as BNP, echocardiograms with Doppler analysis, radionuclide ventriculography, magnetic resonance imaging (MRI), etc.

Multimarker analysis can be used to improve the accuracy of diagnosis and monitoring. For example, abnormal blood concentration levels of angiogenic factors (e.g., soluble FMS-like tyrosine kinase (sFLT-1) and soluble endoglin), placental protein 13 (PP-13), pregnancy-associated plasma protein A (PAPP-A), insulin resistance, apolipoprotein E (apoE), inhibin A, and activin A may be correlated with development and/or diagnosis of preeclampsia. In another example, blood concentrations of galectin-3 (Gal-3) and brain natriuretic peptide (BNP) can be used to diagnose heart failure and to predict the long-term outcome of heart failure (van Kimmenade et al., J. Am. Coll. Cardiol., 48:1217-24 (2006); Sharma et al., Circulation, 110:3121-28 (2004); Lok et al., Eur. Heart J., 28:141, Abstract 1035 (2007)). BNP and its cleavage equivalent amino-terminal proBNP (NT-proBNP) are elevated in heart muscle and in blood during heart failure as a result of high filling pressures of heart chambers and the stretch of cardiac muscle fibers. Other secondary markers that could be used to diagnose heart failure may include non-polypeptidic cardiac markers such as sphingolipid, sphingosine, sphingosine-1-phosphate, dihydrosphingosine and sphingosylphosphorylcholine (see U.S. Pat. No. 6,534,322). When measuring the levels of the above markers, corrections for age and gender may be incorporated to improve the accuracy of diagnosis.

In some embodiments, pregnant or post-partum women may be assessed for a galectin-3 level above or below a threshold level. Alternatively, pregnant or post-partum women may be monitored for changes (e.g., an increase) in galectin-3 levels or activity quantitated from a bodily fluid over time using, for example, an immunoassay. In certain embodiments, a pregnant or post-partum woman may monitor her galectin-3 levels or activity over time, for example, weekly, biweekly (i.e., every other week), monthly, or by trimester. In one embodiment, an initial galectin-3 level or activity may be determined to establish a baseline level or activity, and galectin-3 levels or activity may be monitored subsequently to screen for an increase in level or activity. For example, an initial galectin-3 level or activity may be determined during the first trimester, and galectin-3 levels or activity may be monitored subsequently on a weekly, biweekly, or monthly basis, or during the second and/or third trimester. The woman also may be monitored post-partum. The change in galectin-3 levels in a subsequent sample may be above a minimum threshold. For example, the change in galectin-3 levels in a subsequent sample may be more than about 2 ng/mL, more than about 5 ng/mL, more than about 10 ng/mL, more than about 15 ng/mL, more than about 20 ng/mL, more than about 25 ng/mL, or more than about 30 ng/mL. In some embodiments, the change in galectin-3 levels in a subsequent sample may be between about 2 ng/mL and about 30 ng/mL, between about 2 ng/mL and about 5 ng/mL, between about 5 ng/mL and about 10 ng/mL, between about 10 ng/mL and about 15 ng/mL, between about 15 ng/mL and about 20 ng/mL, between about 20 ng/mL and about 25 ng/mL, between about 25 ng/mL and about 30 ng/mL, or between about 30 ng/mL and about 35 ng/mL.

Pregnant or post-partum woman at risk for developing or having preeclampsia and/or a related condition may be identified by the level of galectin-3 in the pregnant or post-partum woman. For example, a pregnant or post-partum woman at risk for developing or having preeclampsia and/or a related condition may be identified on the basis that the pregnant or post-partum woman has elevated circulatory levels of galectin-3 as compared to healthy non-pregnant women or to women that did not develop preeclampsia or a related condition subject when pregnant. Methods and kits for determining the level of galectin-3 in a subject are disclosed in U.S. patent application Ser. No. 12/608,821, by Muntendam et al., filed on Oct. 29, 2009, and are described in more detail herein.

A pregnant or post-partum woman that may benefit from the methods disclosed herein may be identified on the basis that the circulatory levels of galectin-3 are at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, at least about 20 ng/mL, or at least about 30 ng/mL. In some embodiments, the woman may be identified on the basis that the circulatory levels of galectin-3 are, for example, between about 6 ng/mL and about 19 ng/mL, between about 15 ng/mL and about 20 ng/mL, between about 20 ng/mL and about 25 ng/mL, between about 25 ng/mL and about 30 ng/mL, or between about 30 ng/mL and about 35 ng/mL. In some embodiments, the woman may be identified on the basis that the circulatory levels of galectin-3 are at least about 10% elevated as compared to a standard level, at least about 20% elevated as compared to a standard level, or at least about 50% elevated as compared to a standard level.

In other embodiments, a pregnant or post-partum woman that may benefit from the methods disclosed herein may be identified on the basis that an increase in galectin-3 levels occurs between a first determination of galectin-3 levels and a second determination of galectin-3 levels. For example, the increase in galectin-3 levels may be at least about 2 ng/mL, at least about 5 ng/mL, at least about 10 ng/mL, at least about 15 ng/mL, or at least about 20 ng/mL. In some cases, the woman may be identified on the basis that the absolute increase in galectin-3 levels exceeds a threshold level independent of the time period over which the first and second determinations of galectin-3 levels were made. In other cases, the woman may be identified on the basis that the rate of increase in galectin-3 levels exceeds a threshold level. For example, galectin-3 levels may increase at a rate of about 0.5 ng/mL/week, about 1.0 ng/mL/week, about 1.5 ng/mL/week, about 2 ng/mL/week, about 2.5 ng/mL/week, about 3.5 ng/mL/week, about 4 ng/mL/week, about 4.5 ng/mL/week, about 5 ng/mL/week, about 5.5 ng/mL/week, about 6 ng/mL/week, about 6.5 ng/mL/week, about 7 ng/mL/week, or about 7.5 ng/mL/week.

### Methods of Treatment

In another aspect, methods of treating a pregnant or post-partum woman having preeclampsia and/or a related condition (e.g., eclampsia, HELLP, LVD, HF, or a combination thereof) or at risk of developing preeclampsia and/or a related condition are contemplated. In some embodiments, a composition comprising a compound capable of binding to galectin-3 may be administered to a subject and may at least partially alleviate a symptom of preeclampsia, eclampsia, HELLP, LVD, HF, or a combination thereof. A pregnant or post-partum woman may be identified for treatment according to the methods described above.

For example, in some instances, hypertension may be at least partially relieved. The systolic and/or diastolic blood pressure may be reduced by at least about 5 mm Hg, in some embodiments at least about 10 mm Hg, in some embodiments at least about 15 mm Hg, in some embodiments at least about 20 mm Hg, in some embodiments at least about 25 mm Hg, in some embodiments at least about 30 mm Hg, and in some embodiments at least about 40 mm Hg. As a result of treatment, the woman's blood pressure may be less than about 160/110 mm Hg, less than about 140/90 mm Hg, or less than about 120/80 mm Hg.

Proteinuria may, in some instances, be reduced by at least about 50 mg in a 24 hour collection period, in some embodiments at least about 100 mg in a 24 hour collection period, in some embodiments at least about 150 mg in a 24 hour collection period, in some embodiments at least about 200 mg in a 24 hour collection period, in some embodiments at least about 300 mg in a 24 hour collection period, in some embodiments at least about 400 mg in a 24 hour collection period, in some embodiments at least about 500 mg in a 24 hour collection period, in some embodiments at least about 750 mg in a 24 hour collection period, in some embodiments at least about 1 g in a 24 hour collection period, in some embodiments at least about 1.5 g in a 24 hour collection period, in some embodiments at least about 2 g in a 24 hour collection period, in some embodiments at least about 3 g in a 24 hour collection period, in some embodiments at least about 4 g in a 24 hour collection period, or in some embodiments at least about 5 g in a 24 hour collection period.

An eclampic woman may be relieved of convulsions as a result of galectin-3 inhibition. For example, an eclampic woman may be relieved of convulsions for a period of at least about one week, at least about month, at least about one trimester, at least about 2 trimesters, for the duration of pregnancy, and through the post-partum period.

Low platelet count (i.e., thrombocytopenia) may be at least partially alleviated by inhibiting galectin-3. In some instances, platelet count may be increased by about 10,000 platelets/mm³, in some embodiments by about 20,000 platelets/mm³, in some embodiments by about 50,000 platelets/mm³, in some embodiments by about 75,000 platelets/mm³, in some embodiments by about 100,000 platelets/mm³, in some embodiments by about 150,000 platelets/mm³, and in some embodiments by about 200,000 platelets/mm³. As a result of treatment, a pregnant or post-partum woman may have a platelet count of between 150,000-450,000 platelets/mm³.

High liver enzymes may be at least partially reduced by inhibiting galectin-3. For instance, AST levels may be reduced to less than about 70 U/L and/or LDH levels may be reduced to less than about 600 U/L.

In some embodiments, inhibition of galectin-3 may delay or prevent the onset of preeclampsia and/or related conditions. For example, in some instances, preeclampsia and/or related conditions may be delayed by at least about one month, at least about one trimester, or at least about two trimesters.

For example, in certain embodiments, cardiac fibrosis may be at least partially inhibited. In another embodiment, fractional shortening may be at least partially inhibited from decreasing or may be increased. In some embodiments, left ventricular ejection fraction may be at least partially inhibited from decreasing or may be increased. In other embodiments, right ventricular end diastolic pressure (RVEDP) may be at least partially inhibited from increasing or may be reduced. In yet another embodiment, left ventricle end diastolic pressure (LVEDP) may be at least partially inhibited from increasing or may be reduced. In some embodiments, left ventricular end diastolic volume may be at least partially inhibited from decreasing or may be increased. In other embodiments, left ventricular end systolic volume may be at least partially inhibited from decreasing or may be increased.

In still another embodiment, left ventricle relaxation constant (Tau) may be inhibited from increasing or may be reduced. In another embodiment, cardiac remodeling may be inhibited. In some embodiments, alleviating a symptom may refer to a reduction in the frequency of occurrence of a symptom. In other embodiments, alleviating a symptom may refer to a slowing of the development of a symptom. For example, cardiac fibrosis may occur over a period of time, and treating a subject with compound capable of binding to galectin-3 may slow the progress of cardiac fibrosis.

In some embodiments, fractional shortening and/or left ventricular ejection fraction and/or left ventricular end diastolic volume and/or left ventricular end systolic volume may be increased by at least about 5% or at least about 10%. In some embodiments, LVEDP and/or RVEDP may be decreased by at least about 1 mmHg, at least about 2 mmHg, at least about 3 mmHg, at least about 4 mmHg, or at least about 5 mmHg. In certain embodiments, Tau may be reduced by about 1 msec, about 2 msec, about 3 msec, about 4 msec, or about 5 msec.

In some instances, the rate of progression of a symptom of preeclampsia, eclampsia, HELLP, LVD, and/or HF may be slowed by at least about 5%, at least about 10%, at least about 20%, at least about 30%, at least about 40%, at least about 50%, at least about 60%, at least about 70%, at least about 80%, or at least about 90% when a pregnant or post-partum woman is treated with a compound capable of binding to galectin-3.

In some cases, a compound capable of binding to galectin-3 may reduce the risk of a pregnant or post-partum woman developing preeclampsia, eclampsia, HELLP, LVD, and/or HF. In some embodiments, the risk may be reduced by at least about 5%, at least about 10%, at least about 15%, at least about 20%, at least about 30%, at least about 40%, at least about 50%, at least about 60%, at least about 70%, at least about 80%, at least about 90%, at least about 95%, at least about 99% as compared to an afflicted untreated pregnant or post-partum woman. In some embodiments, treating a pregnant or post-partum woman may reduce the pregnant or post-partum woman's risk of developing preeclampsia, eclampsia, HELLP, LVD, and/or HF to that of normal risk.

In some embodiments, the activity of galectin-3 in a subject (e.g., a pregnant or post-partum woman) may be determined. For instance, the activity of galectin-3 in a subject may be determined as part of a therapeutic regimen. For example, a composition comprising a compound capable of binding to galectin-3 may be administered to a subject (e.g., a pregnant or post-partum woman) and the activity of galectin-3 in the subject may be determined. Such a regimen may be advantageous, for instance, for determining properties such as the proper dosage of the composition, the pharmacokinetics of the composition, the efficacy of the composition, and the like. An activity of galectin-3 may be determined by any method described herein or known to one of ordinary skill in the art. In some embodiments, determining the activity of galectin-3 may comprise determining the fraction of galectin-3 in a biological sample bound to the compound. For instance, an antibody assay may be used where the antibody binds to unbound galectin-3 but does not bind to galectin-3 bound to an inhibitor.

In one aspect, a pregnant or post-partum woman may be given personalized nutritional advice to treat or prevent preeclampsia and/or related conditions. This personalized nutritional advice may include, for example, the identification of a food, supplement, or other ingestible that decreases the in vivo activity of galectin-3. For example, the woman may be advised to ingest foods naturally containing a pectin, or supplements containing a pectin, such as a modified citrus pectin. In one embodiment, a computer is programmed to receive at least two indicators relating to a pregnant or post-partum woman's health, galectin-3 level, diet, identity, and/or health insurance and, in response to these indicators, to transmit data indicative of one or more dietary options effective to impact galectin-3 activity in vivo.

In certain embodiments, galectin-3 levels and/or other biomarkers (such as BNP) can be measured in a pregnant or post-partum woman taking a galectin-3 inhibitor and can be compared to a previous galectin-3 concentration measured in the patient. In some instances, an increase or decrease in galectin-3 concentration relative to one or more previous galectin-3 concentrations in the pregnant or post-partum woman may be an indication that the pregnant or post-partum woman is responding or not responding to galectin-3 inhibitor therapy. Marker levels can be monitored over time, such as in samples obtained from a pregnant or post-partum woman at weekly, biweekly, monthly, or trimester intervals.

Preeclampsia and/or related conditions (e.g., eclampsia, HELLP, LVD, and/or HF) in a pregnant woman are generally cured by delivering the woman's baby, although in some cases one or more of these conditions may continue or first appear post-partum. In some embodiments, it may be preferable to deliver the woman's baby as a treatment for or for prevention of preeclampsia and/or related conditions. For example, galectin-3 levels in a bodily fluid from the pregnant woman that exceed a minimum threshold or that increase by a minimum amount or rate over a period of time may indicate that the woman is a candidate for preterm delivery of the baby.

In some embodiments, delivery of the woman's baby may be performed by caesarian section or by inducing labor. Induction of labor may be accomplished using any suitable technique. For example, a labor-inducing compound may be administered to the pregnant woman. Such compounds include oxytocin, prostaglandins (e.g., dinoprostone), misoprostal, analogs and derivatives thereof, and pharmaceutically acceptable salts and prodrugs thereof. In another embodiment, a physical method may be used to induce labor. For instance, ripening of the cervix may be induced mechanically (e.g., by using a balloon catheter), which can lead to induction of labor. In other examples, stripping or sweeping an amniotic membrane of the pregnant woman or performing an amniotomy on the pregnant woman may be used to induce labor.

### Compositions

In some embodiments, an anti-galectin-3 therapy may comprise administering a compound that is capable of binding (e.g., inhibiting) galectin-3. Any suitable compound may be used. For example, in certain embodiments, the compound may be a carbohydrate, a protein (e.g., an antibody or antibody fragment), a nucleic acid (e.g., an aptamer), or a small molecule.

In some embodiments, the compound may be a carbohydrate. For example, the compound may be a polysaccharide, a disaccharide, a monosaccharide, a pectin, a naturally-occurring carbohydrate, a synthetic carbohydrate, and the like. Pectins are polysaccharides found in the cell walls of terrestrial plants. In some embodiments, a pectin may be a full-length pectin, e.g., a pectin that has not been subjected to fragmentation. In other embodiments, the pectin may be a pectin fragment. In some instances, a pectin may be linear. In other instances, a pectin may be branched. In some cases, the pectin may be a homogalacturonan, a substituted galacturonan, or a rhamnogalacturonan. In some embodiments, a pectin may comprise galactose, xylose, apiose, glucose, arabinose, rhamnose, uronic acid (e.g., galacturonic acid) and/or mannose residues. In some embodiments, a pectin may be a mixture of chemical species. For example, a pectin may comprise a molecular weight distribution of polysaccharide chains. In some instances, a pectin may comprise two or more polysaccharides of different chemical composition.

In some cases, a pectin may be a rhamnogalacturonan pectin. For example, the rhamnogalacturonan may be a rhamnogalacturonan I pectin or a rhamnogalacturonan II pectin. A rhamnogalacturonan I pectin may have, in some embodiments, a backbone of repeating galacturonic acid-rhamnose disaccharides (e.g., α-D-galacturonic acid-(1,2)-α-L-rhamnose). In some cases, rhamnogalacturonan II may have a backbone that is essentially all galacturonic acid residues (e.g., D-galacturonic acid). In some embodiments, at least some of the backbone residues may be substituted with pendant side groups of saccharide residues. In some embodiments, a side group may comprise xylose, apiose, glucose, arabinose, or mannose.

In some embodiments, a pectin may be obtained from a natural source. For instance, in some instances, a pectin may be obtained from a plant source. Non-limiting examples of plant sources include fruits (e.g., apples, guavas, quince, pears, plums, gooseberries, oranges, lemons, grapefruits, other citrus fruits, cherries, grapes, strawberries, and the like) and vegetables (e.g., sugar beets, potatoes, and carrots), although any suitable source may be utilized. In some embodiments, a pectin may be obtained from citrus peel. In other embodiments, a pectin may be obtained from apple pomace. In some embodiments, a pectin may be a swallow root pectic polysaccharide, Hemidesmus pectic polysaccharide, black cumin pectic polysaccharide, Andrographis pectic polysaccharide, citrus pectic polysaccharide, or modified swallow root pectic polysaccharide.

As discussed above, a galectin-3 inhibitor may be a carbohydrate, such as a monosaccharide, a disaccharide, a trisaccharide, a polysaccharide, or analogs or derivatives thereof. Any suitable carbohydrate may be used. In some embodiments, the galectin-3 inhibitor may comprise galactose. In some embodiments, the galectin-3 inhibitor may comprise glucose, galactose, fucose, arabinose, arabitol, allose, altrose, gulose, galactosamine, hammelose, lyxose, mannose, mannitol, mannosamine, ribose, rhamnose, threose, talose, xylose, uronic acids thereof, and combinations thereof. Non-limiting examples of carbohydrates include lactose; LacNAc; Gal-β-1,4-GlcNAc-β-1,3-Gal-β1,4-Glc; Gal-β-1,3-GlcNAc-β-1,3-Gal-β-1,4-Glc; Gal-β-1,4-GlcNAc-β-1,3-Gal; Gal-β-1,4-GlcNAc-β-1,2-(Gal-β-1,4-GlcNAc-β-1,6)-Man; Me-β-LacNAc; Gal-β-1,4-GlcNAc-β-1,2-(Gal-β-1,4-GlcNAc-β-1,4)-Man-α-1,3)-(Gal-β-1,4-GlcNAc-β-1,2-(Gal-β-1,4-GlcNAc-β-1,6)-Man-α-1,6)-Man; Gal-β-1,4-Fru; Gal-β-1,4-ManNAc; Gal-α-1,6-Gal; Me-β-Gal; GlcNAc-β-1,3-Gal; GlcNAc-β-1,4-GlcNAc; Glc-β-1,4-Glc; and GlcNAc; where Gal is galactosyl, Glc is glucosyl, Man is mannosyl, Fm is fructosyl, NAc is N-acetyl, and Me is methyl.

In some embodiments, an ingestible composition may comprise a compound capable of inhibiting galectin-3. For example, the ingestible composition may be a foodstuff. In some cases, the foodstuff may contain a pectin. For example, the foodstuff may be a fruit and/or vegetable product, such as a baked good, a beverage, a mixture of raw and/or cooked fruits and/or vegetables, and the like. In certain embodiments, a foodstuff may be fortified with a compound capable of binding to galectin-3. In some cases, the foodstuff may be fortified with an amount of a compound capable of binding to galectin-3 that is sufficient to have a therapeutic (e.g., anti-hypertensive, anti-proteinuric, anti-hemolytic, hepatoprotective, hepatotherapeutic, anti-thrombocytopenic, cardiotherapeutic, and/or cardioprotective) effect on a pregnant or post-partum woman. Such an approach may be particularly advantageous for improving a foodstuff having non-therapeutically relevant amounts or essentially no amount of a galectin-3 inhibitor.

Additional inhibitors of galectin-3 include nucleic acids, such as antisense nucleic acids and nucleic acid aptamers. Antisense nucleic acids refers to a polynucleotide or peptide nucleic acid capable of binding to a specific DNA or RNA sequence and inhibiting galectin-3 expression. In some embodiments, an antisense nucleic acid may be targeted to a region of the gene encoding galectin-3 in a cell.

In some embodiments, a galectin-3 antibody may be used as an inhibitor of galectin-3. In some cases, the antibody may be selective for an epitope present in galectin-3.

Further description of compositions for inhibiting galectin-3 in a subject are disclosed in U.S. Provisional Application Ser. No. 61/490,049, by Muntendam, filed on May 25, 2011.

In some embodiments, a composition may comprise a plurality of active agents. For example, a composition may include a first active agent that is a compound capable of binding galectin-3 and a second active agent (e.g., an active pharmaceutical ingredient). In some embodiments, a compound capable of binding galectin-3 may be combined with an active agent (e.g., an active pharmaceutical ingredient) suitable for the treatment of preeclampsia, eclampsia, HELLP, LVD, HF, a combination thereof, and/or a symptom thereof (e.g., hypertension, proteinuria, convulsions, seizures,. Non-limiting examples of active agents include angiotensin-converting enzyme (ACE) inhibitors, antiplatelet agents, angiotensin II receptor blockers, beta blockers, calcium channel blockers, diuretics, vasodilators, digitalis preparations, statins, anticonvulsants (e.g., magnesium salts, such as magnesium sulfate), and steroids (e.g., corticosteroids).

In some embodiments, an inhibitor of galectin-3 may have a minimum inhibitory concentration of less than about 1 mg/mL, less than about 500 micrograms/mL, less than about 200 micrograms/mL, less than about 100 micrograms/mL, less than about 50 micrograms/mL, less than about 20 micrograms/mL, less than about 10 micrograms/mL, less than about 5 micrograms/mL, or less than about 1 microgram/mL. In some cases, an inhibitor of galectin-3 may have a minimum inhibitory concentration between about 1 microgram/mL and about 1 mg/mL, between about 1 microgram/mL and about 500 micrograms/mL, between about 1 microgram/mL and about 100 micrograms/mL, between about 5 micrograms/mL and about 500 micrograms/mL, between about 5 microgram/mL and about 100 micrograms/mL, between about 1 microgram/mL and about 50 micrograms/mL, or between about 1 microgram/mL and about 10 micrograms/mL. Inhibitors may be identified, for example, by screening compounds suspected of having galectin-3 binding properties. For example, affinity chromatography using a chromatography resin comprising galectin-3 may be used to capture compounds displaying galectin-3 binding activity. Subsequently, liquid chromatography and mass spectrometry may be used to identify the compounds captured during the affinity chromatography step. One of ordinary skill in the art would readily contemplate other methods and assays for screening compounds suspected of having galectin-3 binding properties.

In some embodiments, binding of a compound to galectin-3 may inhibit an activity of galectin-3. For instance, binding of a compound to galectin-3 may inhibit an interaction between galectin-3 and a biological target, for example, a protein-protein interaction between galectin-3 and another protein, such as a receptor. As discussed above, galectin-3 has been shown to play a role in a variety of cellular process, including cell-cell adhesion, cell-matrix interactions, phagocytosis, cell cycle, apoptosis, angiogenesis, and mRNA splicing. In some cases, inhibition of galectin-3 may inhibit one or more of these processes. However, it should be understood that inhibition of galectin-3 may inhibit other processes as well including inflammation, fibrosis, activation of fibroblasts, organ remodeling, and the like. As galectin-3 has been shown to function through both intracellular and extracellular actions, binding of a compound to galectin-3 may inhibit an intracellular action, an extracellular action, or both an intracellular action and an extracellular action.

In one aspect, inhibition of galectin-3 may be used to treat preeclampsia or a related condition. In some embodiments, inhibition of galectin-3 may be used to treat preeclampsia, eclampsia, HELLP, LVD, HF, or a combination thereof. This list is not meant in any way to be limiting, and other diseases and conditions may be treated as well in a pregnant or post-partum woman. In some embodiments, inhibition of galectin-3 may be used to reduce the risk of developing preeclampsia, eclampsia, HELLP, LVD, HF, or a combination thereof.

In some cases, a compound capable of binding galectin-3 may inhibit an activity of galectin-3 by at least about 5%, at least about 10%, at least about 15%, at least about 20%, at least about 30%, at least about 40%, at least about 50%, at least about 60%, at least about 70%, at least about 80%, or at least about 90%.

In some embodiments, a compound capable of binding galectin-3 may reduce the expression level of galectin-3 by at least about 5%, at least about 10%, at least about 15%, at least about 20%, at least about 30%, at least about 40%, at least about 50%, at least about 60%, at least about 70%, at least about 80%, or at least about 90%.

### Pharmaceutical Formulations

Disclosed compositions may be administered to patients (e.g., pregnant or post-partum women) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound may be administered orally, subcutaneously, topically, parenterally, vaginally, by inhalation spray, or rectally in dosage unit formulations containing conventional non-toxic pharmaceutically acceptable carriers, adjuvants, and vehicles. Parenteral administration may include subcutaneous injections, intravenous or intramuscular injections, or infusion techniques.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a partial or total alleviation of symptoms, is achieved.

In another aspect, pharmaceutical compositions comprising a compound capable of binding galectin-3 as disclosed herein formulated together with a pharmaceutically acceptable carrier are provided. In particular, the present disclosure provides pharmaceutical compositions comprising a compound capable of binding galectin-3 as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions may be used in the form of a pharmaceutical preparation, for example, in solid, semisolid, or liquid form, which contains one or more of the compounds, as an active ingredient, in admixture with an organic or inorganic carrier or excipient suitable for external, enteral, or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another aspect, enteral pharmaceutical formulations including a disclosed pharmaceutical composition comprising a compound capable of binding to galectin-3, an enteric material; and a pharmaceutically acceptable carrier or excipient thereof are provided. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that may be used.

Advantageously, kits are provided containing one or more compositions each including the same or different monomers. Such kits include a suitable dosage form such as those described above and instructions describing the method of using such dosage form to treat a disease or condition. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### EXAMPLES

The invention is further illustrated by the following example. The example is provided for illustrative purposes only, and is not to be construed as limiting the scope or content of the disclosure in any way.

### Example 1

### Association of Blood Plasma Galectin-3 Level with Gestational Preeclampsia

### Methods

### Study Population

A clinical study was conducted as follows. Venous maternal blood plasma was collected from 65 pregnant women diagnosed with preeclampsia. Venous maternal blood samples were also collected from 131 pregnant women who were not diagnosed with preeclampsia at the time of blood collection; these subjects are referred to herein as “control subjects.” For each of the 65 pregnant women diagnosed with preeclampsia, either one or two control subjects were recruited, with control subjects being matched to the respective preeclampsia patient based on the parameters of maternal age (matched to the nearest year) and gestational age (matched to the nearest month).

### Biochemical and Clinical Measurements

The concentration of the galectin-3 protein in each blood plasma specimen was determined using a commercially available assay (BG Medicine Inc., Waltham Mass.), according to the manufacturer's instructions for use.

### Results

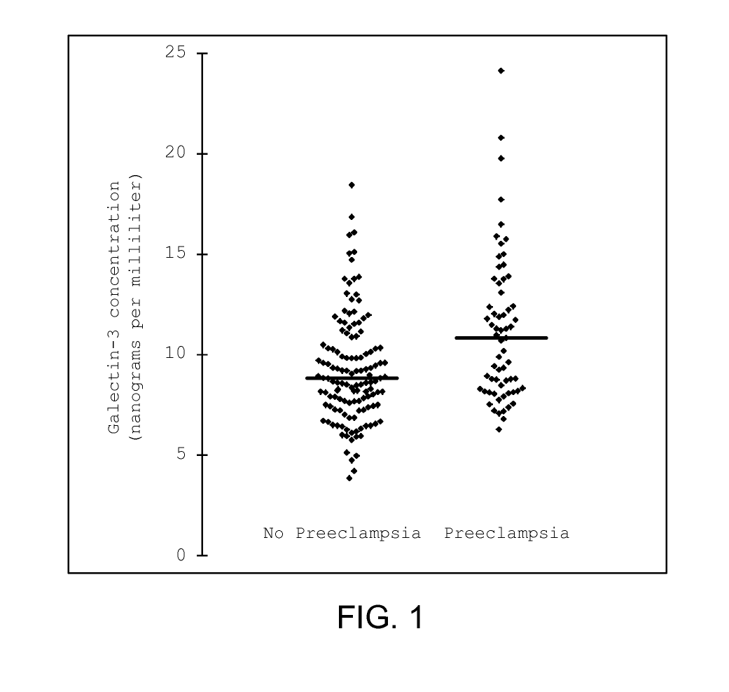

FIG. 1 displays the distribution of blood plasma galectin-3 levels in the 65 pregnant women diagnosed with preeclampsia and the 131 pregnant women who were not diagnosed with preeclampsia. Each data point represents one subject. One value of 46.0 ng/mL in the “Preeclampsia” group is not shown. The horizontal lines among the data points in each group denote the median value.

The distribution of galectin-3 in the two groups of pregnant women is characterized in Table 1 below.

TABLE 1
Distribution of galectin-3 in study populations.

| | Galectin-3 Concentration | |
|---|---|---|
| | (nanograms per milliliter) | |
| | No Preeclampsia | Preeclampsia |
| | (N = 131) | (N = 65) |
| Minimum | 3.9 | 6.3 |
| Maximum | 18.4 | 46.0 |
| 1st Quartile | 7.5 | 8.3 |
| Median | 8.8 | 10.8 |
| 3rd Quartile | 10.3 | 13.6 |
| Mean | 9.2 | 11.7 |
| Standard deviation | 2.7 | 5.6 |

Statistical comparison of the distribution of galectin-3 levels between the group of women diagnosed with preeclampsia and the control subjects was performed using basic linear regression. This analysis yielded a coefficient (comparing women diagnosed with preeclampsia to control subjects) of +2.505, with an associated P-value of 0.0000354. In addition, a linear mixed statistical model that accounted for matching of controls based on maternal age and gestational age was evaluated. This analysis yielded a coefficient (comparing women diagnosed with preeclampsia to control subjects) of +2.51, with an associated P-value less than 0.0001.

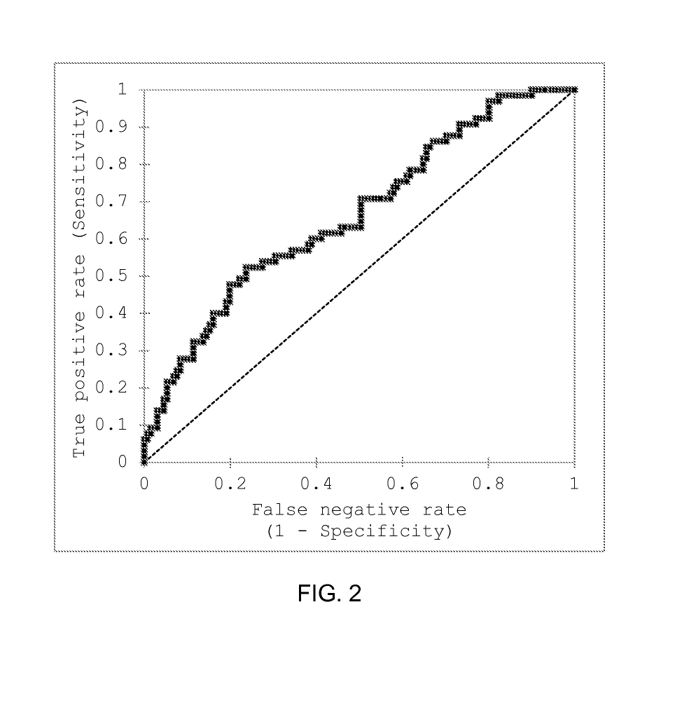

Further, a receiver-operating characteristic curve was constructed using galectin-3 level as the predictor variable. FIG. 2 shows the receiver-operating characteristic curve for the study. The area under the receiver-operating characteristic curve was found to be 0.664, with 95% confidence interval of 0.584 and 0.744. The difference between the area under the receiver-operating characteristic curve and the value of 0.5 (which would indicate no discrimination) was 0.164, with a 95% confidence interval of 0.084 and 0.244. The observed z value for the difference between the area under the receiver-operating characteristic curve and the value of 0.5 was 4.014, and the associated critical z value was 1.960, corresponding to a two-tailed p-value less than 0.0001 at an alpha value of 0.05.

As such, it is determined that levels of galectin-3 in the blood plasma of pregnant women diagnosed with preeclampsia are higher than levels in pregnant women not diagnosed with preeclampsia.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1.-29. (canceled)

30. A method of monitoring development or progression of preeclampsia in a pregnant woman, the method comprising measuring a galectin-3 level in a sample from the pregnant woman, thereby to determine the presence or absence of a galectin-3 level indicative of the development or progression of preeclampsia.

31. A method of monitoring development or progression of elevated liver enzymes and low platelets in a pregnant or post-partum woman, the method comprising measuring a galectin-3 level in a sample from the pregnant or post-partum woman, thereby to determine the presence or absence of a galectin-3 level indicative of the development or progression of elevated liver enzymes and low platelets.

32. A method of monitoring development or progression of left ventricle dysfunction in a pregnant or post-partum woman, the method comprising measuring a galectin-3 level in a sample from the pregnant or post-partum woman, thereby to determine the presence or absence of a galectin-3 level indicative of the development or progression of left ventricle dysfunction.

33. (canceled)

34. The method of claim 30, further comprising measuring a subsequent galectin-3 level in a subsequent sample from the pregnant woman, thereby to detect a change in galectin-3 levels.

35. The method of claim 34, wherein the change in galectin-3 levels is an increase in galectin-3 levels over time.

36. The method of claim 30, wherein the galectin-3 level is more than 6 ng/mL.

37. The method of claim 30, wherein the galectin-3 level is more than 30 ng/mL.

38. The method of claim 34, wherein the change in galectin-3 levels is more than 2 ng/mL.

39. The method of claim 34, wherein the change in galectin-3 levels is more than 10 ng/mL.

40.-66. (canceled)
